(19) United States
(12) Patent Application Publication
(10) Pub. No.: US 2019/0314541 A1
(43) Pub. Date: Oct. 17, 2019

(54) AIR FRESHENER DISPENSER FOR CAR

(71) Applicant: ANA ISABEL RODRIGUEZ-BARBERO, Madrid (ES)

(72) Inventor: ANA ISABEL RODRIGUEZ-BARBERO, Madrid (ES)

(21) Appl. No.: 16/355,400

(22) Filed: Mar. 15, 2019

(30) Foreign Application Priority Data
Apr. 13, 2018 (ES) U 201830525

(51) Int. Cl. A61L 9/14; B60H 3/00

(52) U.S. Cl. CPC A61L 9/14; A61L 2209/13; B60H 3/0007

(57) ABSTRACT

Improved air freshener dispenser for car comprising a rigid air freshener container; a dispensing nozzle longitudinally oriented and coupled to an upper end of the air freshener container such that it emits a dose of air freshener every time the user presses said nozzle downwards; and an absorbing element surrounding an outlet orifice of the dispensing nozzle such that all the air freshener emitted by said nozzle impregnates said absorbing element.

### OBJECT OF THE INVENTION

The present invention belongs to the field of air freshener dispensers.

The object of the present invention is a new air freshener dispenser that doses of the air freshener better and maintains the fragrance longer.

### PRIOR ART OF THE INVENTION

Nowadays, a plurality of types of air freshener dispensers designed for providing a pleasant fragrance in any room are known.

FIG. 1 shows a particular type of dispenser (100) formed by an air freshener flexible container (101) having an elongated shape with an end comprising a tearable area (102) for opening a hole for emitting the air freshener. This dispenser (100) may further comprise a flat part (103) made of an absorbent material and fixed to the container (101) at the end where the hole is located, such that when the tearable area (102) is broken, the flat part (103) is in a position adjacent said hole. FIG. 1 shows a view of an air freshener (100) of this type where all decorative elements usually fitted to the product for sale are eliminated.

In order to use this prior art dispenser (100), the user first tears the tearable area (102) located at the upper end of the container (101) (represented by a broken line in FIG. 1), thus creating an outlet hole in the container (101). Then, the user presses the container (101) anywhere along its length, thus causing the air freshener to exit through the hole. The air freshener emitted through the hole impregnates the part (103) made of an absorbent material, and therefore the fragrance lasts longer in the surrounding environment.

A drawback of this type of dispensers is that possibly a portion of the air freshener exiting through the hole in the container is not absorbed by the part made of an absorbent material, and said portion of air freshener spills around the container and in the hands of the user. In this respect, account must be made that this type of air freshener usually is hanged in a vertical position, for example from a rear view mirror inside a car. Accordingly, it is not possible to ensure that all the air freshener exiting through the hole ends up in the part made of an absorbent material.

Another drawback of these dispensers is that controlling the quantity of air freshener emitted by the manual pressure of the user is not easy. That is, each user, or even the same user at different moments, presses the container with a difference strength, and thus the quantity of air freshener exiting through the hole changes in quantity and injection force.

### DESCRIPTION OF THE INVENTION

The present invention discloses a new air freshener dispenser for car that solves the aforementioned drawbacks. This new dispenser mainly comprises the following elements: an air freshener container; a dispensing nozzle; and an absorbent element. Next, each of these elements is disclosed in greater detail.

### a) Air Freshener Container

The air freshener container is, unlike the containers used in the prior art dispensers, rigid.

Regarding the shape, the container may have any shape as long as it has a closed cavity for receiving the air freshener. However, according to a preferred embodiment of the invention, the container may have an elongated shape, and more preferably an elongated prismatic shape. The prism may have any transversal cross-section, although preferably the cross-section is chosen between circular, elliptical, oval, and rectangular. Any polygonal shape could have rounded or chamfered edges.

Regarding the volume, the container may have any volume providing the dispenser with a reasonable duration. For example, the volume may be between approximately 2 ml and 10 ml.

### b) Dispensing Nozzle

The dispensing nozzle is fitted to an upper end of the air freshener container such that a dose of air freshener is emitted every time the user presses said nozzle downwards. Additionally, the nozzle is longitudinally oriented with respect to the man direction of the container, that is, it is oriented upwards in the natural use position of the dispenser.

The dispensing nozzle is of the type user in the prior art for dispensing cologne or perfume. This type of nozzle is based on a spraying element connected with the inside of the container by means of a duct. When the user pushes down on the spraying element and overcomes the force of a spring, the spraying element moves downwards. When the user stops pushing, the spring urges the sprayer upwards, and said sprayer absorbs a predetermined dose of cologne or perfume that is emitted towards the surrounding environment.

Using this type of nozzles instead of the dispensing mechanism of the prior art dispensers allows for a much more precise dosing of the air freshener. Indeed, using the dispenser type shown in FIG. 1, each time the user presses the flexible container a different quantity of air freshener is emitted. On the contrary, using a nozzle of the present type, the quantity of air freshener emitted by each push is constant and known.

### c) Absorbing Element

The absorbing element surrounds an outlet hole of the dispensing nozzle such that all the air freshener emitted through said nozzle impregnates said absorbing element.

This configuration is advantageous in that spilling the air freshener, as in prior art dispensers of the type shown in FIG. 1, is prevented. Air freshener spilling is not only unpleasant because the user's hands are stained, but they also may cause permanent marks on the dashboard of a car due to their corrosive characteristics.

In principle, the absorbing element may have any shape as long as it surrounds completely the outlet end of the dispensing nozzle such that all the air freshener impregnates said absorbing element. However, in a particularly preferred embodiment of the invention, the absorbing element is formed by two parallel plates coupled to each other that enclose the outlet hole of the nozzle therebetween.

This configuration is advantageous in that the flat plates are easy to produce and transport, while at the same time having a large evaporation surface for the air freshener.

In another preferred embodiment of the invention, an upper end of the absorbing element comprises a hole for hanging. This hole allows for hanging the dispenser of the invention, for example from the rear view mirror of a vehicle.

The dispenser disclosed solves the drawbacks of the prior art, since the possibility of air freshener spills is prevented, and it further allows for a better dosing of the air freshener.

### BRIEF DESCRIPTION OF THE FIGURES

FIG. 1 shows an air freshener dispenser according to the prior art.

FIG. 2 shows a perspective view of a first exemplary air freshener dispenser according to the present invention.

FIG. 3 shows a perspective side view of the exemplary air freshener dispenser of FIG. 2

FIG. 4 shows a view of a second exemplary air freshener dispenser according to the present invention.

FIG. 5 shows a view of the second exemplary air freshener dispenser of FIG. 5 having a decorative element attached thereto.

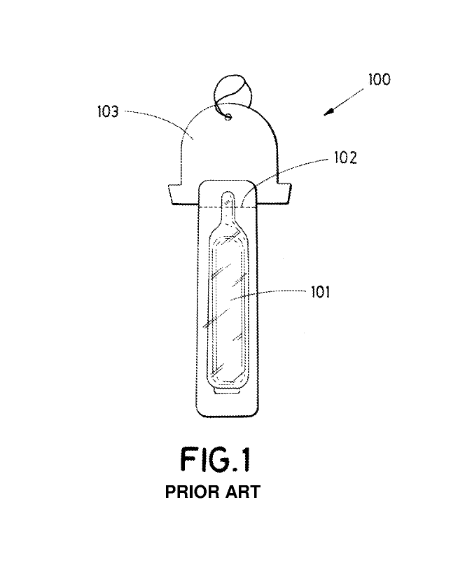

### PREFERRED EMBODIMENT OF THE INVENTION

An exemplary air freshener dispenser (1) according to the present invention is now disclosed in connection with the attached drawings.

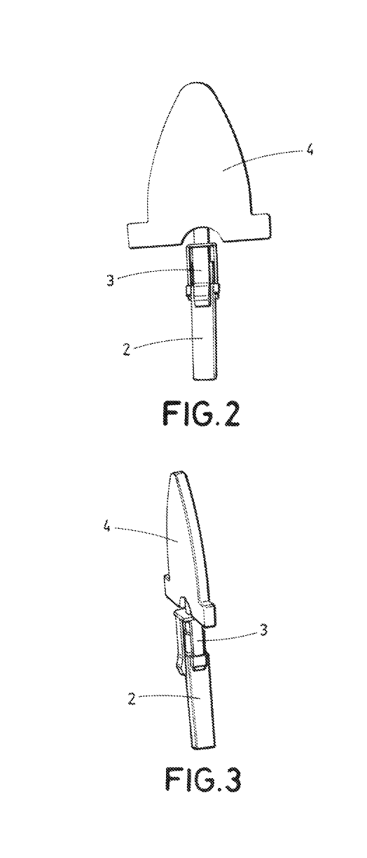

FIGS. 2 and 3 show two perspective views of an air freshener dispenser (1) formed by a rigid container (2) connected at an end thereof to a dispensing nozzle (3), the nozzle having an end embedded in an absorbent element (4) formed by two essentially flat cellulose plates joined to each other. In this present example, the container (2) has the shape of a prism of essentially square cross-section having rounded edges. The nozzle (3) has a lateral protrusion designed for the user to push longitudinally downwards along the main direction of the container (2) for causing the emission of the air freshener. At and end of the nozzle (3) opposite to that where the container (2) is fitted, that is, at an upper end of the nozzle (3), the outlet hole (31) of the nozzle (3) through which emission of the air freshener takes place is located. This outlet hole (31) is completely embedded in the absorbing element (4). The absorbing element (4) is made of cellulose.

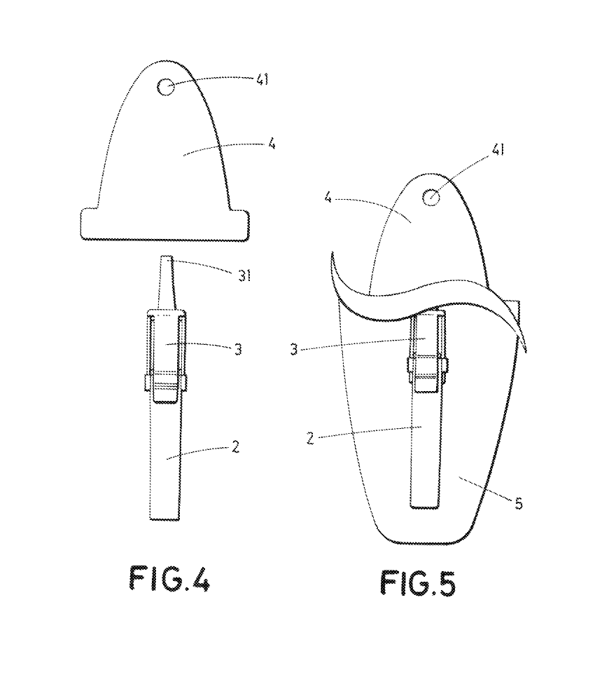

FIGS. 4 and 5 show the aspect of the air freshener dispenser (1) when ready for sale to the public. FIG. 4 shows the unit formed by the container (2) and the nozzle (3) separated from the absorbing element (4). FIG. 5 shows the assembled unit having a decorative plate (5) for making it more pleasant for the public. In these figures, the hole (41) at the upper end of the absorbing element (4) for hanging the dispenser (1), for example from the rear view mirror of a vehicle, is shown.

1. Improved air freshener dispenser (1) for car, characterized by comprising:
a rigid air freshener container (2);
a dispensing nozzle (3) longitudinally oriented and coupled to an upper end of the air freshener container (2) such that it emits a dose of air freshener every time the user presses said nozzle (3) downwards; and
an absorbing element (4) surrounding an outlet orifice (31) of the dispensing nozzle (3) such that all the air freshener emitted by said nozzle (3) impregnates said absorbing element (4).

2. Air freshener dispenser (1) according to claim 1, where the absorbing element (4) is formed by two parallel plates joined to each other and enclosing therebetween the outlet hole (31) of the nozzle (3).

3. Air freshener dispenser (1) according to claim 2, where an upper end of the absorbing element (4) comprises a hole (41) for hanging.

4. Air freshener dispenser (1) according to claim 3, where the absorbing element (4) is made of cellulose.

5. Air freshener dispenser (1) according to claim 4, where the container (2) has an elongated shape.

6. Air freshener dispenser (1) according to claim 5, where the container (2) has an elongated prism shape.

7. Air freshener dispenser (1) according to claim 6, where the container (2) having an elongated prism shape has a cross section chosen from the following list: circular, elliptical, oval, squared, rectangular.

8. Air freshener dispenser (1) according to claim 7, where the container (2) has a volume of between 2 ml and 10 ml.

9. Air freshener dispenser (1) according to claim 1, where an upper end of the absorbing element (4) comprises a hole (41) for hanging.

10. Air freshener dispenser (1) according to claim 1, where the absorbing element (4) is made of cellulose.

11. Air freshener dispenser (1) according to claim 1, where the container (2) has an elongated shape.

12. Air freshener dispenser (1) according to claim 1, where the container (2) has a volume of between 2 ml and 10 ml.
